(19) United States
(12) Patent Application Publication
(10) Pub. No.: US 2020/0222117 A1
(43) Pub. Date: Jul. 16, 2020

(54) ARTICULATING SURGICAL INSTRUMENTS AND METHODS OF DEPLOYING THE SAME

(71) Applicant: Medrobotics Corporation, Raynham, MA (US)

(72) Inventors: Brett Zubiate, Duxbury, MA (US); Thomas J. Calef, Bridgewater, MA (US); Gabriel A. Johnston, Raynham, MA (US); Ian Joseph Darisse, Brighton, MA (US); Michael S. Castro, Plymouth, MA (US); Arnold Oyola, Northborough, MA (US); Kevin Gilmartin, Boston, MA (US); Joseph A. Stand, III, Holden, MA (US); R. Maxwell Flaherty, Auburndale, FL (US); J. Christopher Flaherty, Auburndale, FL (US)

(21) Appl. No.: 16/571,822

(22) Filed: Sep. 16, 2019

Related U.S. Application Data
(63) Continuation of application No. 14/759,020, filed on Jul. 2, 2015, now abandoned.
(60) Provisional application No. 61/751,498, filed on Jan. 11, 2013.
(60) Provisional application No. 61/818,878, filed on May 2, 2013.
(60) Provisional application No. 61/825,297, filed on May 20, 2013.
(60) Provisional application No. 61/909,605, filed on Nov. 27, 2013.
(60) Provisional application No. 61/921,858, filed on Dec. 30, 2013.

(51) Int. Cl. A61B 18/22; A61B 34/30; A61B 1/05; A61B 34/00

(52) U.S. Cl. CPC A61B 18/22; A61B 34/30; A61B 2018/20361; A61B 34/70; A61B 2018/2238; A61B 1/051

(57) ABSTRACT

A robotic introducer system comprises a first assembly comprising a cable control assembly configured for use in a plurality of medical procedures, a second assembly comprising a distal link extension assembly configured for fewer uses than the first assembly, and a third assembly comprising an articulating probe assembly, coupled between the first and second assemblies and configured for fewer uses than the second assembly.

### RELATED APPLICATIONS

This application is a Continuation Application of U.S. Ser. No. 14/759,020, filed Jul. 2, 2015, which is a 371 of International Patent Application No.: PCT/US2014/010808, filed Jan. 9, 2014, which claims the benefit of U.S. Provisional Application No. 61/751,498, filed Jan. 11, 2013, the content of which is incorporated herein by reference in its entirety.

This application claims the benefit of U.S. Provisional Application No. 61/818,878, filed May 2, 2013, the content of which is incorporated herein by reference in its entirety.

This application claims the benefit of U.S. Provisional Application No. 61/825,297, filed May 20, 2013, the content of which is incorporated herein by reference in its entirety.

This application claims the benefit of U.S. Provisional Application No. 61/909,605, filed Nov. 27, 2013, the content of which is incorporated herein by reference in its entirety.

This application claims the benefit of U.S. Provisional Application No. 61/921,858, filed Dec. 30, 2013, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US12/54802, filed Sep. 12, 2012, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US12/70924, filed Dec. 20, 2012, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US12/32279, filed Apr. 5, 2012, the content of which is incorporated herein by reference in its entirety.

This application is related to U.S. patent application Ser. No. 14/008,775, filed Sep. 30, 2013, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US13/54326, filed Aug. 9, 2013, the content of which is incorporated herein by reference in its entirety.

This application is related to U.S. patent application Ser. No. 11/630,279, filed Dec. 20, 2006, published as U.S. Patent Application Publication No. 2009/0171151, the content of which is incorporated herein by reference in its entirety.

### TECHNICAL FIELD

The present inventive concepts generally relate to the field of surgical instruments, and more particularly, to articulating surgical instruments and methods of deploying the same.

### BACKGROUND

As less invasive medical techniques and procedures become more widespread, medical professionals, such as surgeons, may require articulating surgical tools to perform less invasive medical techniques and procedures from outside the human body. Surgical tools, such as endoscopes and other types of tools, typically include expensive electronic components such as cameras and lighting assemblies.

### SUMMARY

In one aspect, provided is a robotic introducer system, comprising a first assembly, a second assembly, and a third assembly. The first assembly comprises a cable control assembly. The first assembly is constructed and arranged for use in a plurality of medical procedures. The second assembly comprises a distal link extension assembly, the second assembly constructed and arranged for fewer uses than the first assembly. The third assembly is coupled between the first and second assemblies. The third assembly comprises an articulating probe assembly to which the distal link extension assembly is removably coupled, and which is controlled by the cable control assembly. The third assembly is constructed and arranged for fewer uses than the second assembly.

In an embodiment, the first assembly further comprises a console system.

In an embodiment, the console system comprises a monitor for displaying images related to a medical procedure of the plurality of medical procedures.

In an embodiment, wherein the console system comprises a human interface device (HID).

In an embodiment, the first assembly comprises a base unit to which the third assembly is coupled.

In an embodiment, the cable control assembly is constructed and arranged to control a movement of the articulating probe assembly.

In an embodiment, the first assembly comprises a brace that attaches the first assembly to at least one of a floor, table or other supporting object.

In an embodiment, the first assembly comprises a handle that permits an operator to move the first assembly relative to the at least one of the floor, table or other supporting object.

In an embodiment, the first assembly is not sterilized for use in the plurality of medical procedures.

In an embodiment, the first assembly is coupled to at least two different second assemblies.

In an embodiment, the second assembly comprises at least one tool guide tube.

In an embodiment, the system further comprises at least one tool constructed and arranged to be slidingly received by the at least one tool guide tube.

In an embodiment, the at least one tool comprises a tool selected from the group consisting of: suction device; ventilator; light; camera; grasper; laser; cautery; clip applier; scissors; needle; needle driver; scalpel; RF energy delivery device; cryogenic energy delivery device; and combinations thereof.

In an embodiment, the at least one tool is positioned at a patient to perform a medical procedure on the patient.

In an embodiment, the medical procedure comprises a transoral surgery procedure.

In an embodiment, the transoral surgery procedure comprises a resection at or near at least one of a base of a tongue, tonsils, a base of a skull, a hypopharynx, a larynx, a trachea, an esophagus, a stomach, or a small intestine.

In an embodiment, the medical procedure comprises at least one of a single or multiport transaxilla, thoracoscopic, pericardial, laparoscopic, transgastric, transenteric, transanal, or transvaginal procedure.

In an embodiment, the single or multiport transaxilla procedure comprises a laryngectomy.

In an embodiment, the single or multiport thoracoscopic procedure comprises a mediastinal nodal dissection.

In an embodiment, the single or multiport pericardial procedure comprises measuring and treating arrhythmias.

In an embodiment, the single or multiport single or multiport laparoscopic procedure comprises a revision of bariatric lap-band procedures.

In an embodiment, the single or multiport transgastric or transenteric procedure comprises at least one of a cholecystectomy or a splenectomy.

In an embodiment, the single or multiport transanal or transvaginal procedure comprises at least one of a hysterectomy, oophorectomy, cystectomy or colectomy.

In an embodiment, the at least one tool guide tube comprises an outer guide tube and an inner guide tube that is slidingly received by the outer guide tube.

In an embodiment, the at least one tool guide tube is coupled to the distal link extension assembly.

In an embodiment, the distal link extension assembly comprises at least one side port, and in an embodiment, each tool guide tube of the at least one tool guide tube is coupled to a side port of the at least one side port.

In an embodiment, the distal link extension assembly further comprises a first side port coupled to a first tool guide tube and a second side port coupled to a second tool guide tube.

In an embodiment, the at least one side port comprises a working channel.

In an embodiment, the system further comprises a tool extending through the working channel.

In an embodiment, the system further comprises a lighting fiber extending through the working channel that transmits light from a light source.

In an embodiment, the lighting fiber is for a single use.

In an embodiment, the lighting fiber is reusable.

In an embodiment, the distal link extension assembly comprises a camera assembly.

In an embodiment, the distal link extension assembly comprises a distal link body having a central opening that is configured to receive the camera assembly.

In an embodiment, the distal link body comprises a first side port and a second side port extending therefrom.

In an embodiment, each of the first and second side ports comprises a working channel for receiving a tool.

In an embodiment, the camera assembly comprises a lens assembly that generates images of objects related to at least one of the medical procedures.

In an embodiment, the camera assembly comprises a calibration adjustment nut in communication with the lens assembly for providing focus adjustments to a lens of the camera assembly.

In an embodiment, the camera assembly comprises a camera sensor that processes the images.

In an embodiment, the lens assembly comprises a lens barrel comprising an interior region that houses and provides for a precise alignment of one or more optics.

In an embodiment, the lens assembly comprises one or more spacers positioned between two or more of the one or more optics for providing axial and/or radial alignment of the two or more optics.

In an embodiment, the one or more optics include one or more lenses.

In an embodiment, the one or more optics include a polarizing or filtering lens that controls glare, reflected lights from instruments, or other undesirable effects.

In an embodiment, the one or more optics filter infrared (IR) or visible wavelengths.

In an embodiment, the filtering lens is constructed and arranged to allow wavelengths to pass ranging from 400 to 700 nm.

In an embodiment, the filtering lens is constructed and arranged to block infrared wavelengths.

In an embodiment, the filtering lens is constructed and arranged to block ultraviolet wavelengths.

In an embodiment, the filtering lens is constructed and arranged to block LISA laser wavelengths.

In an embodiment, the lens assembly is constructed and arranged for more uses than the second assembly.

In an embodiment, the camera assembly comprises a working channel that extends through the camera assembly.

In an embodiment, the camera assembly is constructed and arranged for more uses than the second assembly.

In an embodiment, the distal link extension assembly further comprises a lighting assembly that outputs electromagnetic radiation.

In an embodiment, the electromagnetic radiation comprises light.

In an embodiment, the lighting assembly comprises a diffusing lens for providing a uniform field of view.

In an embodiment, the lighting assembly comprises a printed circuit board comprising a light source.

In an embodiment, the light source comprises an electron stimulated light source.

In an embodiment, the electron stimulated light source comprises at least one of an electron stimulated luminescence light source, an incandescent light source, an electroluminescent light source, or a gas discharge light source.

In an embodiment, the incandescent light source comprises an incandescent light bulb.

In an embodiment, the gas discharge light source comprises a fluorescent lamp.

In an embodiment, the electroluminescent light source comprises a light-emitting diode (LED).

In an embodiment, the LED is constructed and arranged to produce 1-100 lumens.

In an embodiment, the LED is constructed and arranged to provide a color temperature range between 2700K and 7000K.

In an embodiment, the LED is a multicolor LED.

In an embodiment, the light source comprises a laser light source.

In an embodiment, the laser light source comprises a vertical cavity surface emitting laser (VCSEL).

In an embodiment, the light source comprises at least one optical fiber, which is constructed and arranged to transmit light to and from the lighting assembly.

In an embodiment, the lighting assembly comprises a light source coupled to an optical fiber. In an embodiment, the optical fiber is coupled to a distal lens. In an embodiment, the electromagnetic radiation is output from the light source through the optical fiber to the distal lens.

In an embodiment, the working channel of the distal link extension assembly is constructed and arranged to receive at least one tool.

In an embodiment, the at least one tool comprises a tool selected from the group consisting of: suction device; ventilator; light; camera; grasper; laser; cautery; clip applier; scissors; needle; needle driver; scalpel; RF energy delivery device; cryogenic energy delivery device; and combinations thereof.

In an embodiment, the second assembly further comprises an introduction device that is constructed and arranged to slidingly receive the articulating probe assembly.

In an embodiment, the articulating probe assembly is slidingly positioned in the introduction device.

In an embodiment, the second assembly comprises at least one tool guide tube constructed and arranged to slidingly receive a tool.

In an embodiment, the at least one tool guide tube is directly anchored to the introduction device.

In an embodiment, the second assembly further comprises a base coupled to the introduction device.

In an embodiment, the second assembly further comprises at least one inner guide tube slidingly received by the at least one tool guide tube and anchored to the distal link extension assembly.

In an embodiment, the second assembly further comprises a guide tube support.

In an embodiment, the second assembly further comprises at least one outer guide tube coupled between the guide tube support and the base.

In an embodiment, the guide tube support comprises a dogbone connector.

In an embodiment, the guide tube support comprises a tool entrance opening in communication with the tool guide tube.

In an embodiment, the system further comprises an uninterrupted tool path from the tool entrance opening, the tool guide tube, and a tool exit port of the distal link extension assembly.

In an embodiment, the base comprises a collar that surrounds at least a portion of the introduction device.

In an embodiment, the collar extends in a lateral direction relative to a direction of extension of the introduction device.

In an embodiment, the collar has first and second openings and in an embodiment, first and second outer guide tubes of the tool guide tube are coupled to one side of the first and second openings, and first and second inner guide tubes extend from the first and second outer guide tubes, respectively, at a second side of the first and second openings.

In an embodiment, the second assembly is cleaned, disinfected and/or resterilized between uses.

In an embodiment, the second assembly is coupled to at least two third assemblies over the lifetime of the second assembly.

In an embodiment, the second assembly is coupled to each of the at least two third assemblies in different procedures.

In an embodiment, the articulating probe assembly comprises a plurality of links that are constructed and arranged to facilitate a manipulation of the articulating probe assembly.

In an embodiment, the distal link extension assembly of the second assembly is coupled to a distal connecting link at a distal end of the plurality of links of the articulating probe assembly.

In an embodiment, the third assembly is constructed and arranged for a single use.

In an embodiment, the articulating probe assembly comprises at least one multi-link inner probe and a multi-link outer probe. In an embodiment, the inner and outer probes are steerable by the cable control assembly.

In an embodiment, the third assembly comprises a probe feeder that is coupled to the first assembly for controlling a movement of the articulating probe assembly.

In another aspect, provided is a robotic introducer system, comprising: an articulating probe assembly; a distal link extension assembly coupled to a distal end of the probe assembly; at least one side port extending from the distal link extension assembly, the at least one side port constructed and arranged to receive a tool; and an optical assembly at the distal link extension assembly. The optical assembly comprises a lens providing a first field of view for a user; and an optical redirector that provides a second field of view for the user, the second field of view including a view of the tool received at the at least one side port.

In an embodiment, the second field of view comprises the at least one side port.

In an embodiment, the optical assembly is removably coupled to the probe assembly.

In an embodiment, the optical redirector comprises at least one of a mirror or a prism.

In an embodiment, the at least one side port comprises a first side port constructed and arranged to receive a first tool and a second side port constructed and arranged to receive a second tool.

In an embodiment, the system further comprises a second optical redirector that provides a third field of view for the user.

In another aspect, provided is a robotic introducer system, comprising: an articulating probe assembly; and a distal link extension assembly coupled to a distal end of the articulating probe assembly, the distal link extension assembly including a base; a body movably positioned in the base; an optical lens coupled to the body; and a plurality of body articulating cables extending along the probe assembly and the base that moves the body to change a field of view of the lens when a force is applied to at least one of the cables.

In an embodiment, the articulating probe assembly and the body are independently controllable.

In an embodiment, the articulating probe assembly comprises a plurality of probe links, and in an embodiment, the distal link extension assembly is adjacent a distal link of the plurality of probe links.

In an embodiment, the articulating probe assembly comprises at least one steering cable that terminates at the distal link of the plurality of probe links.

In an embodiment, the at least one steering cable and the plurality of body articulating cables are independently controllable.

In an embodiment, a lower region of the body is convex.

In an embodiment, the base comprises a concave region into which the convex lower region of the body is positioned.

In an embodiment, the convex lower region of the body is a semi-spherical body portion.

In an embodiment, the convex lower region of the body is a semi-ellipsoidal body portion.

In an embodiment, the concave region is a semi-ellipsoidal cavity portion.

In an embodiment, a lower region of the body is concave, and the base comprises a convex region onto which the concave lower region of the body is positioned.

In an embodiment, the body is ball-shaped.

In an embodiment, the system further comprises a plurality of guide holes, each of the plurality of body articulating cables extending through a guide hole of the plurality of guide holes.

In an embodiment, the articulating probe assembly include a plurality of probe links.

In an embodiment, each of the plurality of probe links comprises a guide hole, and in an embodiment, each of the plurality of guide holes are aligned with each other to receive an articulating body cable.

In an embodiment, the system further comprises a plurality of tubes extending through the plurality of guide holes along the articulating probe assembly that advance and retract with respect to the articulating probe assembly for articulating the probe assembly, a distal end of each of the plurality of tubes coupled to the base.

In an embodiment, the plurality of body articulating cables extend through the plurality of tubes, and move independently of the plurality of tubes.

In an embodiment, the plurality of body articulating cables and the plurality of tubes operate to pan, tilt, or zoom the body.

In an embodiment, the plurality of tubes are spaced equidistantly about the articulating probe assembly.

In an embodiment, the system further comprises a camera assembly positioned in the body, the camera assembly comprising the optical lens.

In another aspect, provided is a method of deploying a robotic introducer system, comprising: providing a first assembly comprising a cable control assembly for use in a plurality of medical procedures; providing a second assembly comprising a distal link extension assembly for fewer uses than the first assembly; coupling a third assembly between the first and second assemblies, the third assembly comprising an articulating probe assembly to which the distal link extension assembly is removably coupled, the third assembly constructed and arranged for fewer uses than the second assembly; and controlling, by the cable control assembly, the articulating probe assembly.

In an embodiment, the method comprises the robotic introducer system including additional features as claimed.

In another aspect, provided is a robotic introducer system as described in reference to the figures.

In another aspect, provided is a method of using a robotics introducer system as described in reference to the figures.

In another aspect, provided is a method of performing a medical procedure as described in reference to the figures.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a perspective view of a robotic introducer system, in accordance with embodiments of the present inventive concepts;

FIG. 2 is a perspective view of the second assembly of FIG. 1, in accordance with an embodiment;

FIG. 3A is a perspective view of the distal link extension assembly of FIGS. 1 and 2, in accordance with an embodiment;

FIG. 3B is an exploded view of the distal link extension assembly of FIG. 3A, in accordance with an embodiment;

FIG. 3C is an exploded view of the lighting assembly of FIG. 3B, in accordance with an embodiment;

FIG. 4A is a perspective view of the camera assembly of FIGS. 3A and 3B, in accordance with an embodiment;

FIG. 4B is an exploded view of the camera assembly of FIGS. 3A, 3B, and 4A, in accordance with an embodiment;

FIG. 5A is a perspective view of the lens assembly of FIGS. 4A and 4B, in accordance with an embodiment;

FIG. 5B is a cross-sectional view of the lens assembly of FIGS. 4A, 4B, and 5A, in accordance with an embodiment;

FIG. 5C is an exploded view of the lens assembly of FIGS. 4A, 4B, 5A and 5B, in accordance with an embodiment;

FIG. 6 is a flowchart illustrating a method for assembling a robotic introducer system to perform an operation, in accordance with an embodiment;

FIG. 7 is a flowchart illustrating a method for assembling a robotic introducer system to perform an operation, in accordance with an embodiment;

FIG. 8 is a cross-sectional view of an optical assembly, in accordance with an embodiment;

FIG. 9 is a view of a display at a console, the display including a displayed image generated from the optical assembly of FIG. 8, in accordance with an embodiment;

FIG. 10 is a cross-sectional view of a robotic introducer system comprising a distal camera assembly, in accordance with an embodiment;

FIG. 11A is a perspective view of the distal end of an articulating probe including a set of attaching elements, in accordance with an embodiment; and

FIG. 11B is a perspective view of the proximal end of a distal link extension assembly including a set of attaching elements that can mate with the attaching elements of the articulating probe of FIG. 11A, in accordance with an embodiment.

### DETAILED DESCRIPTION OF EMBODIMENTS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

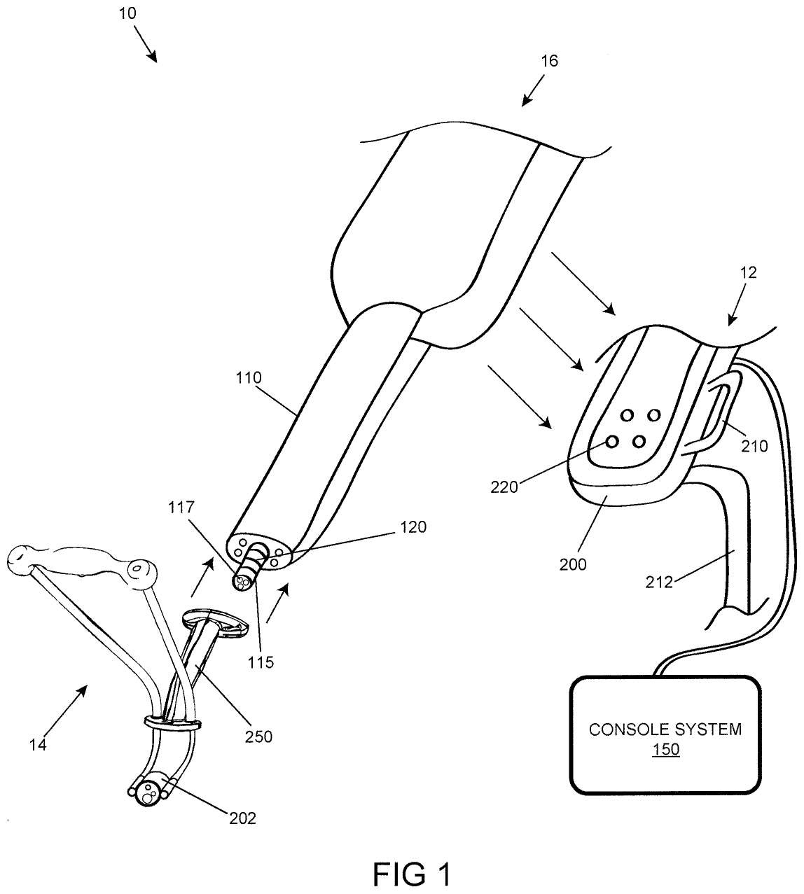

FIG. 1 is a perspective view of a robotic introducer system 10, in accordance with embodiments of the present inventive concepts. The robotic introducer system 10 can be constructed and arranged to perform a medical procedure, such as a transoral robotic surgery procedure. The system 10 may include one or more features of a surgical positioning and support system, for example, described in PCT Application serial number PCT/US2011/044811, filed Jul. 21, 2011, PCT Application serial number PCT/US2012/32279, filed Apr. 5, 2012, and PCT Application No. PCT/US2013/054326, filed Aug. 9, 2013, the contents of each of which are herein incorporated by reference in their entirety.

The robotic introducer system 10 is constructed and arranged to position one or more tools (not shown) for performing a medical procedure on a patient, for example, a transoral robotic surgery procedure or the like, or other surgical procedure that includes inserting one or more tools into a cavity of the patient, or a region of the patient formed by an incision or related opening. A surgical procedure can include one or more transoral procedures, including but not limited to resections at or near the base of a tongue, tonsils, a base of a skull, hypopharynx, larynx, trachea, esophagus and within the stomach and small intestine. Other medical procedures can include but not be limited to single or multiple transaxilla procedures, such as a laryngectomy, single or multiple thoracoscopic procedures, such as a mediastinal nodal dissection, single or multiple pericardial procedures, for example, related to measuring and treating arrhythmias, single or multiple laparoscopic procedures, such as revision of bariatric lap-band procedures, single or multiple transgastric or transenteric procedures, such as a cholecystectomy or splenectomy, and/or single or multiple transanal or transvaginal procedures, such as a hysterectomy, oophorectomy, cystectomy and colectomy.

The robotic introducer system 10 includes a first assembly 12, a second assembly 14, and a third assembly 16. The first assembly 12 is constructed and arranged to be used a plurality of times in one or more medical procedures. The second assembly 14 is constructed and arranged to be used fewer times than the first assembly 12. The third assembly 16 is constructed and arranged to be used in one or more medical procedures, but fewer times than the second assembly 14. In some embodiments, the third assembly 16 is constructed and arranged for a single use. In some embodiments, the third assembly 16 is constructed and arranged for multiple uses, but fewer uses than the second assembly 14.

The term “use” can refer to a use of the first, second, and/or third assembly in one or more procedures for a particular patient. For example, the third assembly 16 can be used to perform one or more medical procedures on one patient, removed from the system 10, and replaced with a different third assembly 16 that is used to perform one or more medical procedures on a different patient. In another example, the third assembly 16 can be used to perform a procedure on one patient, removed from the system 10, and replaced with a different third assembly 16 that is used to perform a different procedure on the same patient.

The first, second, and/or third assemblies 12, 14, 16 can include a processor and a memory for storing program code for performing one or more features and functions described herein. For example, program code for performing a camera calibration such as a gamma correction, or for counting the number of clinical uses of an assembly, can be stored in the memory.

The second and third assemblies 14, 16 are typically sanitized (e.g. cleaned, disinfected and/or sterilized) for each use. Unlike the second and third assemblies 14, 16, in some embodiments, the first assembly 12 is not positioned in an environment that requires sterilization after each use, for example, sterilization that would be required between medical procedures performed on different patients. In other embodiments, one or more portions of first assembly 12 are covered by one or more sterile barriers, such as sterile drape positioned between first assembly 12 and third assembly 16. The second assembly 14 can be sanitized (e.g. cleaned, disinfected and/or sterilized) between uses. In some embodiments, the third assembly 16 is sanitized, typically sterilized, for a single use, and is removed from the first and third assemblies 12, 16, and disposed of, after its single use.

The first assembly 12 includes a base unit 200 comprising a cable control assembly 220 that controls a movement of an articulating probe assembly 120 of the third assembly 16, described below. The base unit 200 can include other elements similar to those described in PCT Application No. PCT/US2012/070924, filed Dec. 20, 2012, the contents of which are incorporated herein by reference in their entirety.

The first assembly 12 includes a base stand 212, or related brace, which attaches the base unit 200 to a floor, a patient operating table, or other supporting object. A handle 210 can extend from the base unit 200 that permits an operator to move the robotic introducer system 10 relative to the supporting structure to which the base stand 212 is coupled, for example, a floor, a patient operating table, etc., before or during a medical procedure, or between different procedures.

The first assembly 12 comprises a console system 150. The console system 150 includes a monitor and a human interface device (HID) (not shown). The monitor may be configured to display images and/or sensor readings from tools or related devices, e.g., cameras, probes, sensors, which are coupled to or otherwise provided with the articulating probe assembly 120, the second assembly 14 and/or one or more other components of the system 10. The console system 150 may further include an input device, such as a keyboard, touch screen, touch pad and/or pointing device, for communicating with elements of the robotic introducer system 10, such as the articulating probe assembly 120.

An operator, such as a surgeon or other medical professional, may control the robotic introducer system 10 via the HID to manipulate or otherwise control the functions and movement of the articulating probe assembly 120, for example, steer, advance, retract or otherwise control the functions and movement of articulating probe assembly 120. The HID may include a hand-operated control device, such as a joystick.

The first assembly 12 can be coupled to one or more different third assemblies 16, for example, over the lifetime of the first assembly. Features of an exemplary third assembly are described at PCT Application No. PCT/US2012/070924, filed Dec. 20, 2012, the contents of which are incorporated by reference above.

The third assembly 16 can be coupled between the first assembly 12 and the second assembly 14, such as a coupling the directions shown by the arrows. The third assembly 16 comprises a probe feeder 110 that is removably coupled between the first assembly 12 and the second assembly 14. The articulating probe assembly 120 of the third assembly 16 is removably coupled to the second assembly 14. The probe feeder 110 can include a carriage, guide rails, cables, gears, and/or other mechanical devices that communicate with the cable control assembly 220 of the base unit 200 of the first assembly 12 to control a movement of the articulating probe assembly 120, and/or one or more tools in communication with the articulating probe assembly 120. For example, the base unit 200 can include motor driven wheels, which engage and drive bobbins, gears, or the like, which in turn can advance and retract a carriage of the probe feeder 110.

The articulating probe assembly 120 can include a plurality of links that are constructed and arranged to facilitate a manipulation of the probe assembly 120, which in turn can guide one or more surgical tools during a medical procedure. The links can be constructed and arranged to form at least one multi-link inner probe (not shown) and a multi-link outer probe, similar to a probe assembly described in PCT Application No. PCT/US2012/032279, filed Apr. 5, 2012, the content of which is incorporated herein by reference above. The inner probe can include a plurality of inner links and the outer probe can include a plurality of outer links. The inner probe and the outer probe can communicate with each other by a plurality of steering cables (not shown), which are steerable by the cable control assembly 220, for example, which can advance or retract the links with respect to one another during manipulation of the articulating probe assembly 120. The steering cables can be used to releasingly tighten to lock or stiffen either or both of the plurality of inner links or the plurality of outer links. Accordingly, the inner probe and the outer probe can be configured in one of a limp mode and a rigid mode so as to facilitate the manipulation of the articulating probe assembly 120. For example, the inner and outer links may be configured in one of the limp mode and the rigid mode via one or more steering cables of the articulating probe assembly 120.

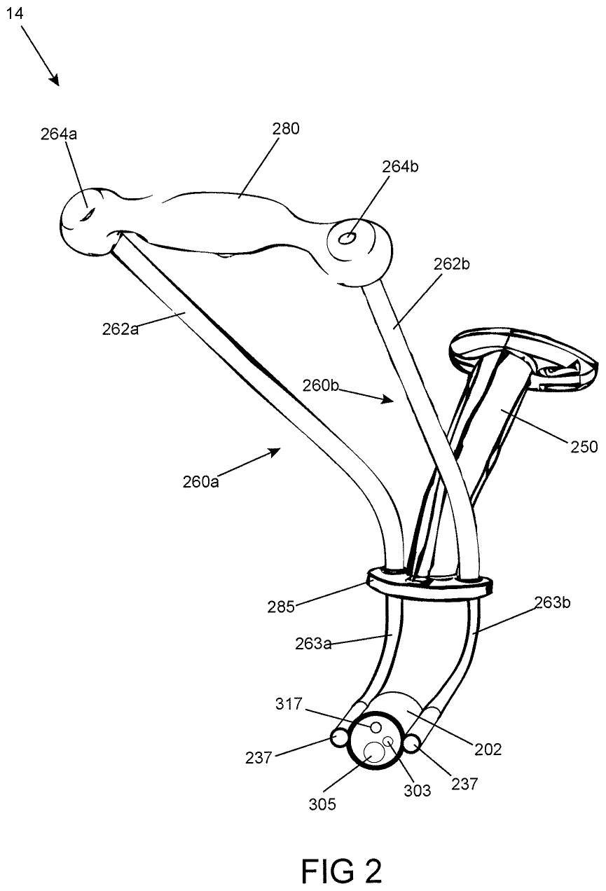

The articulating probe assembly 120 includes a connecting link 115 at a distal end of the outer links, also referred to as a distal link, which is removably coupled to a portion of the second assembly 14, as described in FIG. 2 herein. The connecting link 115 can include one or more working channels 117 for transferring electrical signals and/or tools to the second assembly 14. The working channels 117 may extend through some or all of the articulating probe assembly 120, for example, in a channel between the inner and outer links, from a proximal end to a distal end of the articulating probe assembly 120. The working channels 117 can be aligned with working channels extending through a distal link extension assembly of the second assembly 14, as described herein.

The second assembly 14 includes an introduction device 250 constructed and arranged to slidingly receive the articulating probe assembly 120. The second assembly 14 is also constructed and arranged to position and/or provide support to one or more tools (not shown) for performing a medical procedure on a patient. The second assembly 14 can be coupled over its lifetime to at least two different third assemblies 16, for example, where each third assembly 16 is constructed and arranged to perform a single use, while the second assembly 14 is constructed and arranged for reuse. In an embodiment, the second assembly 14 includes a distal link extension assembly 202 for coupling with the connecting link 115 at the distal end of the articulating probe assembly 120 of the third assembly 16.

FIG. 2 is a perspective view of the second assembly 14, in accordance with an embodiment. As described in FIG. 1, the second assembly 14 comprises an introduction device 250. The second assembly 14 also comprises a first tool guide tube 260a, and a second tool guide tube 260b, also referred to as tool supports. Although two tool guide tubes 260a, 260b (generally, 260) are shown, the second assembly 14 can be constructed and arranged to include more than two tool guide tubes 260 or, alternatively, can include a single guide tube 260. Each tool guide tube 260 is constructed and arranged to slidingly receive a tool or other elongate object used in a medical procedure.

The first tool guide tube 260a can include an outer guide tube 262a and an inner guide tube 263a that is slidingly received by the outer guide tube 262a. The second tool guide tube 260b can include an outer guide tube 262b and an inner guide tube 263b that is slidingly received by the outer guide tube 262b. Accordingly, each of the tool guide tubes 260 can have an inner guide tube 263a, b (generally, 263) that movably extends from the outer guide tube 262a, b (generally, 262), for example, in a telescoping configuration.

At least a portion of each inner guide tube 263 is flexible. To achieve this, an inner guide tube 263 can include one or more hinged sections. At least a portion of each outer guide tube 262 is rigid, with limited or no flexibility. The inner guide tubes 263 can be formed of plastic or related material. Materials can include but are not limited to fluoropolymers (e.g., polytetrafluoroethylene), fluorinated ethylene propylene, polyether block amide, high density polyethylene, low density polyethylene and/or nickel titanium alloy. The inner guide tubes 263 can comprise laser cut tubes, e.g. polymer or metal tubes with cuts placed to provide flexibility, and/or coils or braids of plastic or metal. In some embodiments, an inner guide tube 263 comprises a polytetrafluoroethylene liner. In some embodiments, an inner guide tube 263 comprises a stainless steel coil. In some embodiments, an inner guide tube 263 comprises a coil covered by a polyether block amide. In some embodiments, an inner guide tube 263 comprises varying stiffness along its length.

The second assembly 14 can include a base 285. The base 285 can comprise a collar that surrounds at least a portion of the introduction device 250, and is fixedly attached to the surface of the introduction device 250. The collar can extend in a lateral direction relative to a direction of extension of the introduction device 250. The collar has first and second openings. The outer guide tubes 262 of the tool guide tube 260 can be coupled to one side of the first and second openings, and the inner guide tubes 263 can extend from the first and second outer guide tubes 262, respectively, at a second side of the first and second openings. The first tool guide tube 260a and the second tool guide tube 260b are coupled to the base 285 to maintain a relative position between the first tool guide tube 260a and the second tool guide tube 260b and/or maintain a fixed orientation between the first tool guide tube 260a and the second tool guide tube 260b. The base 285 can also comprise an opening for receiving, and holding in place against, the introduction device 250 and/or an articulating probe, such as probe assembly 120 of system 10, advanced therethrough.

One or more tool guide tubes 260 can rotatably engage the base 285. The tool guide tube 260 can be coupled to the base 285 by a gimbal or other pivoted or ball and joint mechanism (not shown), permitting the tool guide tube 260 to rotate relative to the base 285, for example, allowing for three degrees of freedom between tool guide tube 260 and base 285, which can include two-dimensional (X-Y) movement plus rotation.

In other embodiments, the first and second tool guide tubes 260a, 260b are fixedly coupled to a surface of the introduction device 250 instead of a base, for example, via welding points, adhesives, or other bonding mechanisms. The connection at the introduction device 250 maintains a fixed distance and/or a fixed orientation between the first tool guide tube 260a and the second tool guide tube 260b. In some embodiments, the first and second tool guide tubes 260a and 260b can be rotatably attached to each other and/or a base for maintaining a fixed distance but not a fixed orientation. The first tool guide tube 260a and the second tool guide tube 260b can be fixed in position relative to each other. Accordingly, positions of the first and second tool guide tubes 260a, 260b can be maintained during an operation of the robotic introducer system 10.

The second assembly 14 can include a guide tube support 280 coupled to the first tool guide tube 260a and the second tool guide tube 260b. The guide tube support 280 is constructed and arranged to maintain a relative position between the first tool guide tube 260a and the second tool guide tube 260b. In some embodiments, guide tube support 280 is constructed and arranged to maintain a relative orientation between the first tool guide tube 260a and the second tool guide tube 260b. In an embodiment, the guide tube support 280 includes a dogbone connector, for example, described with reference to PCT Application No. PCT/US2013/054326, filed Aug. 9, 2013, incorporated by reference above. The guide tube support 280 can be removably attached to the tool guide tubes 260a, 260b. Accordingly, in some embodiments, the guide tube support 280 is used with two or more different second assemblies 14, depending on the medical procedure. For example, in a first medical procedure, the guide tube support 280 is attached to a second assembly 14. After the first medical procedure, the guide tube support 280 can be sanitized, and used in a second medical procedure, where the guide tube support 280 is attached to a different second assembly 14.

The guide tube support 280 can comprise a rigid structure. Alternatively, the guide tube support 280 can comprise a malleable or flexible structure. The guide tube support 280 can comprise at least a portion that is flexible. The guide tube support 280 can comprise an operator shapeable structure. The guide tube support 280 can comprise two segments connected by a hinge, such as a butt hinge, a butterfly hinge, a barrel hinge or a hinge comprising a flexible portion positioned between two rigid portions. The guide tube support 280 can comprise a telescopically adjustable structure, such as to allow separation of tool supports 260a and 260b. The guide tube support 280 can comprise two segments connected by a rotatable connector, such as a universal joint.

The guide tube support 280 can be constructed and arranged to be shaped, molded, or the like, such as after the application of heat. The guide tube support 280 can be constructed and arranged to be attachable to at least one of the first tool guide tube 260a or the second tool guide tube 260b. The guide tube support 280 can be constructed and arranged to be detachable to at least one of the first tool guide tube 260a or the second tool guide tube 260b.

The guide tube support 280 comprises a first opening 264a and a second opening 264b (generally 264), each constructed and arranged to operably engage an outer guide tube 262a, 262b of the first and second tool supports 260a, 260b, respectively. The first opening 264a and the second opening 264b can be constructed and arranged to position the first tool guide tube 260a and the second tool guide tube 260b in a non-parallel configuration. At least one of the first opening 264a or the second opening 264b can comprise a funnel-shaped opening, for example, for receiving an outer guide tube 262. In this manner, an uninterrupted tool path can extend from an opening 264 at the guide tube support 280 through a tool guide tube 260 to a tool exit at a side port 237 of the distal link extension assembly.

In embodiments where a tool guide tube 260 is slidably adjustable, thus allowing for a shortening of a portion of the guide tube 260 that attaches to the guide tube support 280, the guide tube support 280 may require adjustability of the distance between connector openings. Depending on the desired relative orientation of one guide tube 260 to the other, parallel or angled, then the adjustability in the guide tube support 280 for the distance between openings can occur along a straight or curved path. The tool guide tube 260 can be locked in a fixed position relative to the base 285. The second assembly 14 can include a locking mechanism (not shown) to lock the at least one tool guide tube 260 in the fixed position. The locking mechanism may be constructed to secure a position of the tool guide tubes 260 with respect to the base 285, thus preventing the tool guide tubes 260 from sliding or otherwise moving axially during movement of the tools by one or more operators.

An outer guide tube 262 can have a funnel-shaped proximal end (not shown). The inner guide tube 263 can likewise have a funnel shaped proximal end (not shown). Either or both funnels can be configured to readily and atraumatically introduce tools to the tool guide tube 260. A funnel shaped proximal end of each tool guide tube 260 can be positioned about an opening 264 in a guide tube support 280. In this manner, an uninterrupted tool path can extend from an opening 264 through a tool guide tube 260 to a tool exit at a side port 237 of second assembly 14.

The introduction device 250 can be constructed and arranged to slidingly receive the articulating probe assembly 120 of the third assembly 16 of FIG. 1, and support, stabilize, and/or guide the articulating probe assembly 120 to a region of interest. The region of interest may be a lumen of a body of a patient, such as a cavity at the patient's head, e.g., a nose or mouth, or an opening formed by an incision. In clinical applications, typical regions of interest can include but not limited to the esophagus or other locations within the gastrointestinal tract, the pericardial space, the peritoneal space, and combinations thereof. The region of interest may alternatively be a mechanical device, a building, or another open or closed environment in which the articulating probe assembly 120 can be used.

In an embodiment, the second assembly 14 includes a distal link extension assembly 202 for coupling with the connecting link 115 at the distal end of the articulating probe assembly 120 of the third assembly 16. The connecting link 115 coupled to the distal link extension assembly 202 provides stability between the second assembly 14 and the third assembly 16, and also permits a transfer of electrical signals, power, light, liquid and/or energy between the distal link extension assembly 202 and the connecting link 115. The distal link extension assembly 202 and the connecting link 115 can comprise multiple elements constructed and arranged to mechanically attach the two components together, such as one or more snaps, threads or magnetic couplers.

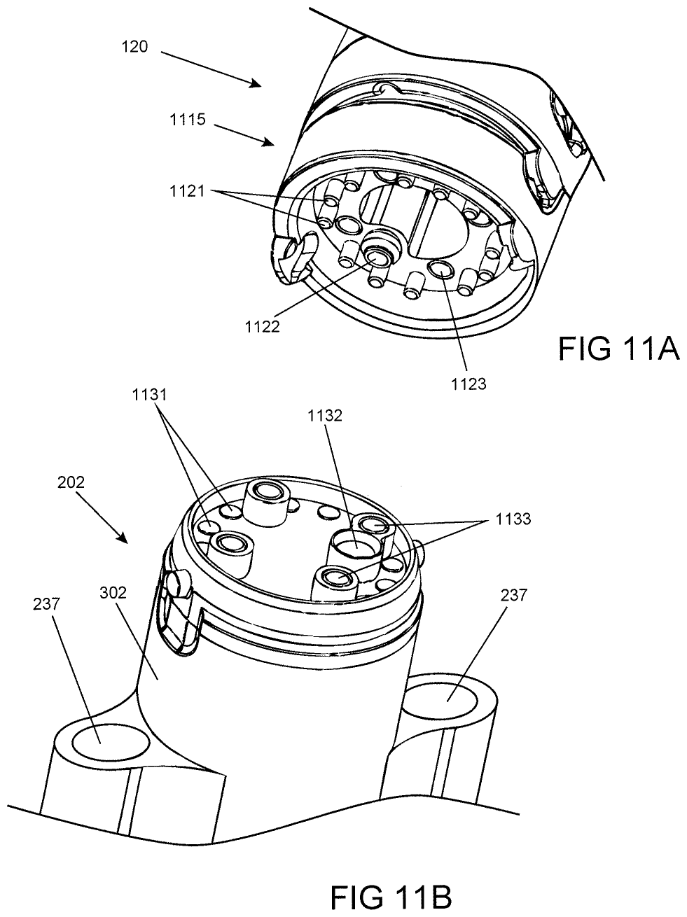

FIG. 11A is a perspective view of the distal end of an articulating probe assembly 120 including a set of attaching elements, in accordance with an embodiment. FIG. 11B is a perspective view of the proximal end of a distal link extension assembly 202 including a set of attaching elements that can mate with the attaching elements of the articulating probe assembly 120 of FIG. 11A, in accordance with an embodiment.

In an embodiment, the articulating probe assembly 120 includes a distal link 1115, also referred to as a distal connecting link. The distal link 1115 can include one or more electrical connectors 1121. The electrical connectors 1121 can comprise frictionally engaging pins, such as pogo pins configured to electrically engage opposing electrical contacts such as one or more electrical contacts 1131 extending from the distal link extension assembly 202.

The distal link 1115 further includes a male connector 1122 constructed and arranged to couple with a female connector 1132 of the distal link extension assembly 202. Mating connectors 1122 and 1132, when coupled together, can extend a working channel 317 (working channel 317 shown in FIG. 2), which can provide electrical signals, wiring, fiber optics, or the like to electrical elements of the distal link extension assembly 202, described herein. In some embodiments, connectors 1122 and 1132 may include fluid tight connectors, for example when a working channel 317 includes an irrigation channel or other fluid transfer channel.

The distal link 1115 and the distal link extension assembly 202 can also include one or more fasteners 1123 and 1133, respectively, for securing the distal link extension assembly 202 to the distal link 1115. One or more fasteners may include fasteners selected from the group consisting of: magnets; snap fit connectors; threaded connectors; or combinations of these. One or more fasteners can be configured to ensure a proper alignment of the distal link 1115 and the distal link extension assembly 202.

Referring again to FIG. 2, at least one side port 237 can extend from an outer surface of the distal link extension assembly 202. In an embodiment, a first side port 237 is coupled to the first tool guide tube 260a and a second side port 237 is coupled to the second tool guide tube 260b. Each side port 237 can provide a guide for an inner guide tube 263. An outer guide tube 262 and/or inner guide tube 263 can be constructed and arranged to guide or otherwise provide a support for a tool shaft so that it can be guided from the guide tube support 280 to a side port 237 extending from the distal link extension assembly 202.

The distal link extension assembly 202 can also include one or more working channels 317 that are aligned with the working channels 117 of the connecting link 115 (working channels 117 shown in FIG. 1). Any number of surgical tools or related accessories may be slidingly received by the working channels 117, 317 and/or the side ports 237, including but not limited to a cameras, light or other radiation sources, cutters, graspers, scissors, energy appliers, suturing assemblies, biopsy removal elements, ventilators, lasers, cautery, clip appliers, scissors, needles, needle drivers, scalpels, RF energy delivery devices, cryogenic energy delivery devices, drug delivery devices, EKG electrodes, pressure sensors, a blood sensors, magnets, heating elements, or combinations thereof As shown in FIG. 2, the distal link extension assembly 202 can include a camera lens 305 and a lighting source 303, such as an LED light source, which can be collocated with at least one working channel 317.

In an embodiment, at least one side port 237 includes a working channel at which a tool is positioned. In another embodiment, a lighting fiber assembly extends through the working channel of the side port 237 for transmitting light from a light source positioned proximal the lighting fiber. The lighting fiber assembly can be steerable, so that light can be directed to a working area. In an embodiment, the lighting fiber assembly can be for a single use. In another embodiment, the lighting fiber assembly can be configured for a plurality of uses.

The second assembly 14 can include at least one fixation point (not shown) for attaching to the introduction device 250, the base 285, the first tool guide tube 260a, second tool guide tube 260b, the guide tube support 280, and/or a combination thereof A brace (not shown) can be attached between a fixation point and an operating room floor, a patient operating table, and/or an articulating probe feeder such as the feeder 110 shown in FIG. 1. The brace can include a clamping device or the like, for clamping to a floor, table or other supporting object. Multiple braces can be coupled to different fixation points. For example, a brace (not shown) can be coupled between a fixation point at the base 285 and a fixation point at the first tool guide tube 260a. Another brace can be attached to the feeder 110 and can be clamped or otherwise attached to a floor, table or other object providing stability.

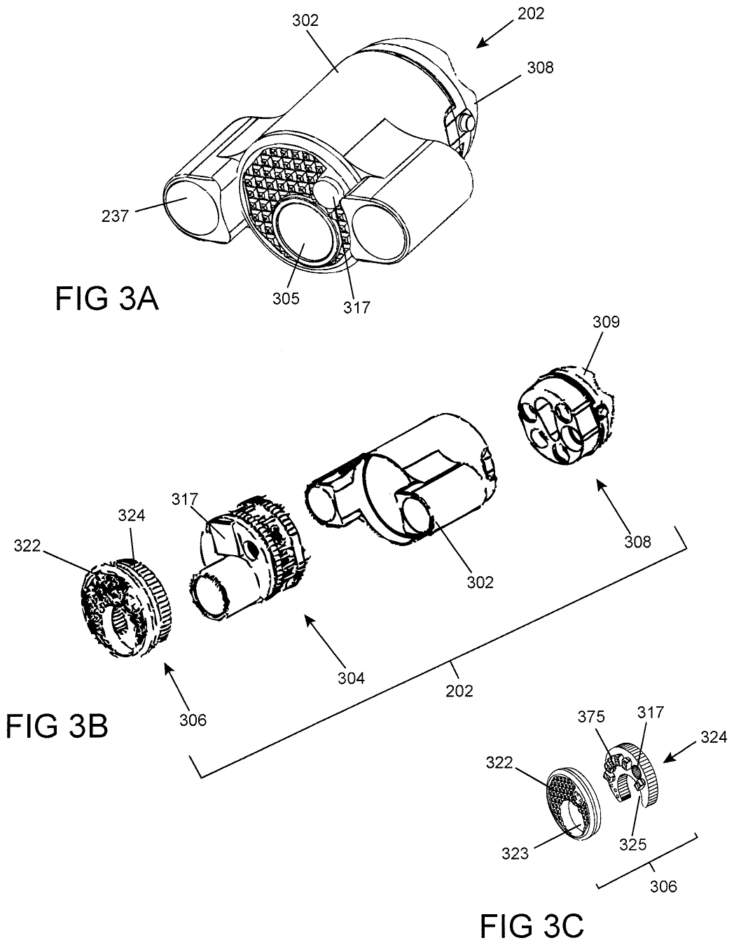

FIG. 3A is a perspective view of the distal link extension assembly 202 of FIGS. 1 and 2, in accordance with an embodiment. FIG. 3B is an exploded view of the distal link extension assembly 202 of FIG. 3A, in accordance with an embodiment. FIG. 3C is an exploded view of a lighting assembly 306 of FIG. 3B, in accordance with an embodiment.

The distal link extension assembly 202 includes a distal link body 302, a camera assembly 304, a lighting assembly 306, and a link connector 308. The distal link body 302 has a central opening that is configured so that the camera assembly 304 and lighting assembly 306 can be removably positioned in the distal link body 302. Some or all of the distal link extension assembly 202 can be removed from the second assembly 14, and replaced, for example, during a resterilization between uses of the second assembly 14. A camera lens 305 and a diffusing lens 322 can be exposed at one end of the distal link body 302. In other embodiments, the camera assembly 304 and/or the lighting assembly 306 are external to the distal link body 302, for example, positioned at the surface of the distal link body 302. The link connector 308 can be coupled to the other end of the distal link body 302. The distal link body 302 can include one or more side ports 237 that extend from an outer surface of the distal link body 302.

The link connector 308 can have a body portion 309 that movably mates with the connecting link 115 at the distal end of the articulating probe assembly 120. For example, the body portion 309 can have a convex portion that is positioned in a cavity in the connecting link 115. Accordingly, the connecting link 115 and the distal link extension assembly 202 can articulate relative to each other during operation.

The lighting assembly 306 is positioned between the camera assembly 304 and a field of view. The lighting assembly 306 includes a diffusing lens 322 or related camera lens filter that diffuses or scatters light produced by the lighting assembly 306, for providing a uniform field of view. The diffusing lens 322 can be coupled to a printed circuit board (PCB) 324 having one or more light sources 375. The light sources 375 may include electron stimulated light sources such as electron stimulated luminescence light sources, incandescent light sources such as incandescent light bulbs, electroluminescent light sources such as light-emitting diodes (LEDs), and gas discharge light sources such as fluorescent lamps, or related sources that produce high power light. An electron stimulated light source can include an electron stimulated luminescence light source, an incandescent light source, an electroluminescent light source, and/or a gas discharge light source. An incandescent light source can include an incandescent light bulb. A gas discharge light source can include a fluorescent lamp.

An LED can be constructed and arranged to produce a predetermined amount of electromagnetic energy, for example, between 1-250 lumens of light. One or more LEDs can be constructed and arranged to provide a color temperature range between 2700K and 7000K A single LED or multiple discrete LEDs providing different forms of light that collectively produce a desired effect. An LED can be constructed and arranged to produce at least one of infrared light or ultraviolet light or other range of frequencies known to those of ordinary skill in the art. An LED can be a multicolor LED. Thus, one or more LEDs with multicolor capabilities can generate a desired color temperature, or be used in conjunction with filters to produce desired emphasis or accentuate certain features/colors/tissue. Multiple LEDs, such as two or more independently controlled LEDs, can display differing colors to produce a desired color, color temperature, or effect.

In other embodiments, a light source 375 includes a laser light source, for example, a vertical cavity surface emitting laser (VCSEL). The laser light source can be excited by use of another laser through an optical fiber or the like to energize a VCSEL, thereby eliminating an electric shock risk from the light source.

The PCB 324 may further include optical fibers, which can be configured to transmit light to and from the articulating probe assembly 120 and/or another component of the robotic introducer system 10. The diffusing lens 322 can include an opening 323. The PCB 324 can likewise include an opening 325. The diffusing lens 322 and the PCB 324 are coupled together so that the diffusing lens opening 323 is aligned with the PCB opening 325 for receiving a camera lens 305 of the camera assembly 304, and so that the diffusing lens 322 is positioned in front of a light source 375, for example, an LED.

In another embodiment, the light source 375 is at a different location than a lens at a distal end of the distal link extension assembly 202. The light source 375 is coupled to an optical fiber or other transmitter, which in turn is coupled to the distal lens. Here, light or other electromagnetic radiation is generated at the light source 375 and transmitted to the distal lens via the optical fiber.

The distal link extension assembly 202 can include at least one working channel 317 that extends through the camera assembly 304 and the link connector 308 to provide electrical signals, wiring, fiber optics, or the like to the lighting assembly 306.

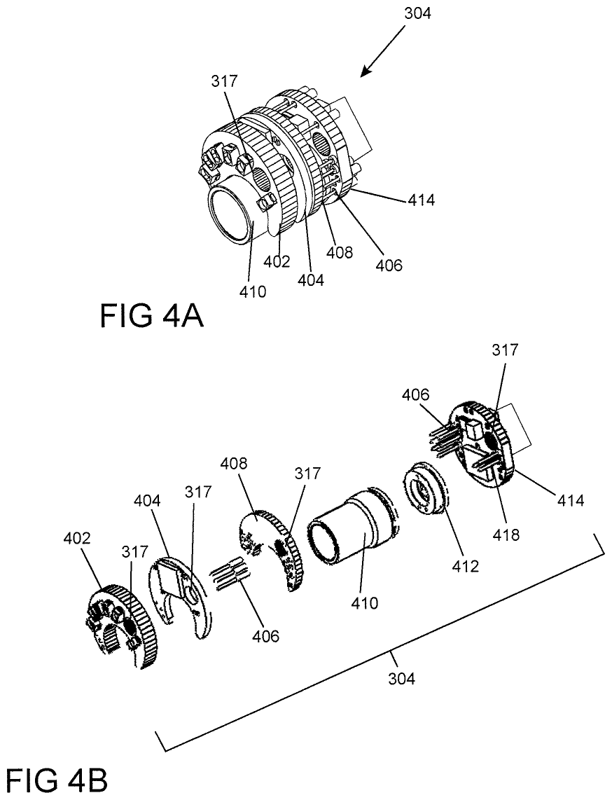

FIG. 4A is a perspective view of the camera assembly 304 of FIGS. 3A and 3B, in accordance with an embodiment. FIG. 4B is an exploded view of the camera assembly 304 of FIGS. 3A, 3B, and 4A, in accordance with an embodiment.

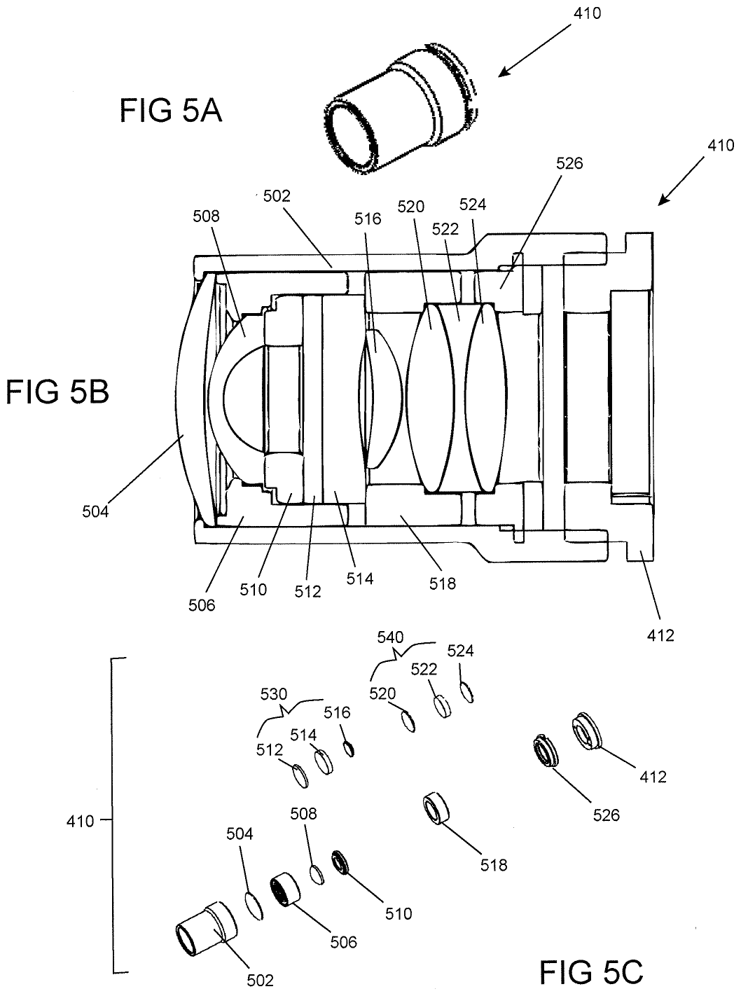

The camera assembly 304 includes a lens assembly 410 that focuses images of objects, which can be detected by a visual camera or other sensor device and transmitted to a console system, for example console system 150 of FIG. 1, stored on a media, or otherwise used in a manner that is well-known to those of ordinary skill in the art. The objects are related to a medical procedure, for example, taken of a patient undergoing a treatment. The lens assembly 410 can be removed from the camera assembly 304, and replaced, for example, during a sanitization, e.g. a resterilization, between uses performed by the second assembly 14. A calibration adjustment nut 412, also referred to as a lens mount, can be threaded into the lens assembly 410 for adjusting a lens focus or calibrating the lens assembly 410, for example, during manufacturing. Additional description of the calibration adjustment nut 412 is provided with respect to FIGS. 5A-5C. A PCB 414 having an image sensor 418 is coupled to one end of the lens assembly 410. The image sensor 418 can include a charge coupled device (CCD), CMOS sensor, or related sensing device for processing an image provided by the lens assembly 410.

The camera assembly 304 can include multiple PCBs, such as a first PCB 402, a second PCB 404, and a third PCB 408. Multiple PCBs can be used to fit necessary imaging, image processing, power and/or other electronic components within a constrained dimension, such as a maximum diameter, while expanding the assembly in a less constrained axial direction. The camera assembly 304 can include a plurality of connecting pins 406 for electrically and/or mechanically coupling the second and third PCBs 404, 408 with each other, and a plurality of connecting pins 406 for electrically and/or mechanically coupling the third PCB 408 and PCB 414 with each other. For example, as illustrated herein, the working channel 317 extends through the camera assembly 304.

FIG. 5A is a perspective view of the lens assembly 410 of FIGS. 4A and 4B, in accordance with an embodiment. FIG. 5B is a cross-sectional view of the lens assembly 410 of FIGS. 4A, 4B, and 5A, in accordance with an embodiment. FIG. 5C is an exploded view of the lens assembly of FIGS. 4A, 4B, 5A, and 5B, in accordance with an embodiment.

The lens assembly 410 includes a lens barrel 502 having an interior region that houses and provides for a precise alignment of one or more optics, spacers, and related elements, each described herein. One of the optics includes a front lens 504, which is fixed in place in the lens barrel 502 by a mounting structure that includes one or more spacers, for example, spacer 506, and/other elements described herein with respect to FIGS. 5A-5C. The lens barrel 502 is constructed and arranged for positioning optics such as the one or more lenses to their required accuracy, while protecting the optics from environmental conditions such as temperature, stress, vibrations, or biological contaminates. The lens barrel 502 can include a seat, for example, a tangential seat, at which the front lens 504 can be radially and/or axially aligned by a tangent contact with respect to an optical surface of the front lens 504. The front lens 504 can collect electromagnetic radiation such as light from a predetermined field of view, for example a field of view between 50° and 135°, such as a field of view of approximately 82°.

The lens assembly 410 can include one or more additional optics such as a polarizing or filtering lens, which can be constructed and arranged to control glare, reduce reflected lights from instruments (e.g. laser flare), or reduce other undesirable effects. One or more lenses described herein can filter one or more wavelengths (e.g. IR or visible light wavelengths) such as to accentuate features, colors, etc., to reduce or eliminate external light, and/or to provide a trigger signal. In an embodiment, a filtering lens can be constructed and arranged to allow particular wavelengths to pass ranging from 400 nm to 700 nm. In an embodiment, the filtering lens can be constructed and arranged to block infrared wavelengths, e.g. wavelengths ranging from 700 nm to 1105 nm. In an embodiment, the filtering lens can be constructed and arranged to block ultraviolet wavelengths ranging from 1 nm to 400 nm. In an embodiment, the filtering lens can be constructed and arranged to block LISA laser wavelengths for example, 2000 nm wavelength.

The spacer 506 provides an axial and/or radial alignment for the meniscus lens 508, the spacer 510, and an aperture/filter assembly 530. The meniscus lens 508 can direct light or other electromagnetic radiation at the camera aperture. Radial and/or axial alignment of the meniscus lens 508 can be established by a tangent contact of the spacer 506 with its optical surface. The spacer 510 provides an axial location for aperture/filter assembly 530, which comprises a filter glass 512, a lens 514, and a lens 516. In some embodiments, the lens 514 is a plano-concave lens (as shown) configured to accept light from the filter glass 512 and direct light into the lens 516. The lens 516 can comprise a meniscus lens (as shown) that is mounted to the lens 514 (e.g. cemented) such that light exiting the lens 514 is directed toward the concave surface of the lens 516. In an embodiment, the filter glass 512 prevents predetermined wavelengths from being transmitted, for example, a 2 μm wavelength. The filter glass 512 can include an opaque coating that creates an aperture to limit an amount of light reaching an image sensor, such as the image sensor 418 of FIG. 4B. The spacer 506 can provide a radial alignment of the filter glass 512. The spacer 510, in particular, a flat surface of the spacer 510, can provide an axial alignment of the filter glass 512. In some embodiments, a radial and axial alignment of the aperture/filter assembly 530 with respect to the filter glass 512 is set during manufacturing.

The spacer 518 can provide an axial and/or a radial alignment for a triplet assembly 540, which comprises a lens 520, a lens 522, and a lens 524. The light exiting lens 516 is directed into a triplet assembly 540. In some embodiments, lens 520 is a convex-convex lens (as shown) which receives the light exiting lens 516 and directs light toward the lens 522. The lens 522 can comprise a concave-concave lens (as shown) which receives the light from the lens 522 and directs light onto the lens 524. The lens 524 can comprise a convex-convex lens (as shown) which receives the light from the lens 522 and directs light towards an image sensor, such as the image sensor 418 of FIG. 4B. The triplet assembly 540 provides color correction and focuses light on the sensor 418. The triplet assembly 540 can set a radial and axial alignment by tangent contact of spacer 518 with an optical surface.

The lens retainer 526 compresses the lens stack together to maintain their respective alignments. Lens retainer 526 can be constructed and arranged to sufficiently compress the multiple lenses of the lens assembly 410. The lens retainer 526 can also provide centering from a rear of the triplet assembly 540 by contacting lens 524 at a tangent with respect to the optical surface.

The lens mount 412, also referred to as a calibration adjustment nut, attaches the lens assembly 410 to the assembly 304 of FIG. 4A. The lens mount 412 is aligned with the sensor 418 by a close tolerance fit rectangular cavity that surrounds the sensor 418, thus providing an accurate alignment of the lens assembly 410 to the sensor 418. The lens mount 412 can include a thread for attaching to the lens assembly 410, allowing focus adjustments to be made, for example, by rotating the lens assembly 410 to achieve an optimal optical distance to the sensor 418.

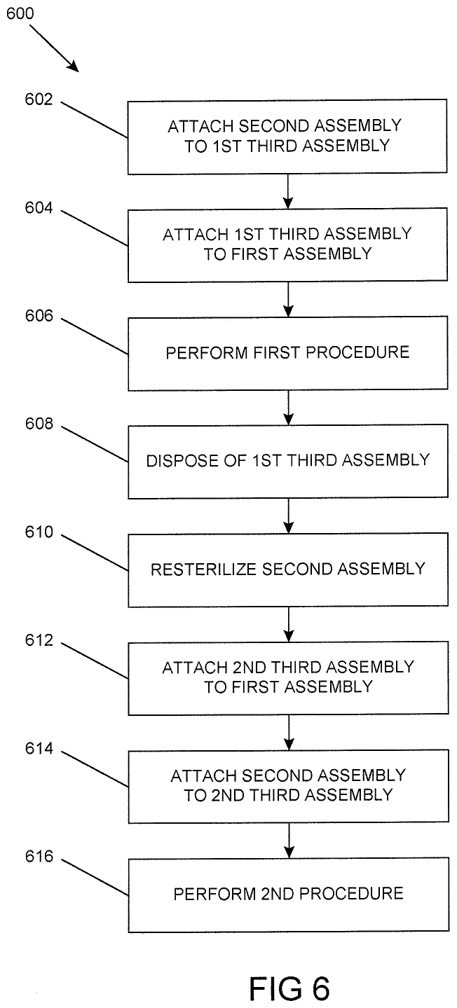

FIG. 6 is a flowchart illustrating a method 600 for assembling a robotic introducer system 10 to perform an operation, in accordance with an embodiment. When describing the method 600, reference is made to FIG. 1. Although the method 600 refers to a sequence of blocks, or steps, the method 600 is not limited to this sequence. In other embodiments, various blocks can be performed in a different order. For example, block 604 can be performed prior to block 602.

At block 602, the second assembly 14 is attached to a third assembly 16 of one or more different third assemblies 16 used with second assembly 14, for example, over its lifetime. This attachment can include extending the connecting link 115 of the articulating probe assembly 120 of the third assembly 16 through the introduction device 250 to the second assembly 14 to the distal link extension assembly 202.

At block 604, the third assembly 16 is attached to the first assembly 12. This manipulation can include attaching a carriage, guide rails, cables, gears, and/or other mechanical devices (not shown) of the probe feeder 110 of the third assembly 16 to the cable control assembly 220 of the first assembly 12. Accordingly, the robotic introducer system 10 is operational by attaching the first assembly 12, the second assembly 14, and the third assembly 16 to each other. In some embodiments, a sterile barrier such as a sterile drape is placed between first assembly 12 and third assembly 16.

At block 606, a first procedure can be performed by the robotic introducer system 10, for example, a medical procedure, such as a transoral robotic surgery procedure.

At block 608, the third assembly 16 is removed from the robotic introducer system 10. In some embodiments, the third assembly 16 is constructed for a single use, and is sanitized (e.g. sterilized) one time prior to that single use. In these embodiments, after the single use, i.e., the first procedure, is completed, the third assembly 16 is disposed of.

At block 610, the second assembly 14 can be sanitized (e.g. sterilized) after the first procedure, and prior to a subsequent procedure performed by the robotic introducer system 10.

At block 612, another third assembly that is different than the third assembly 16 referred to at blocks 602, 604, 606, and 608 is attached to the first assembly 12.

At block 614, the sanitized second assembly 14 is attached to the new third assembly 16. Accordingly, the robotic introducer system 10 is operational by attaching the first assembly 12, the second assembly 14, and the third assembly 16 to each other.

At block 616, a second procedure can be performed by the robotic introducer system 10, for example, a medical procedure, such as a transoral robotic surgery procedure.

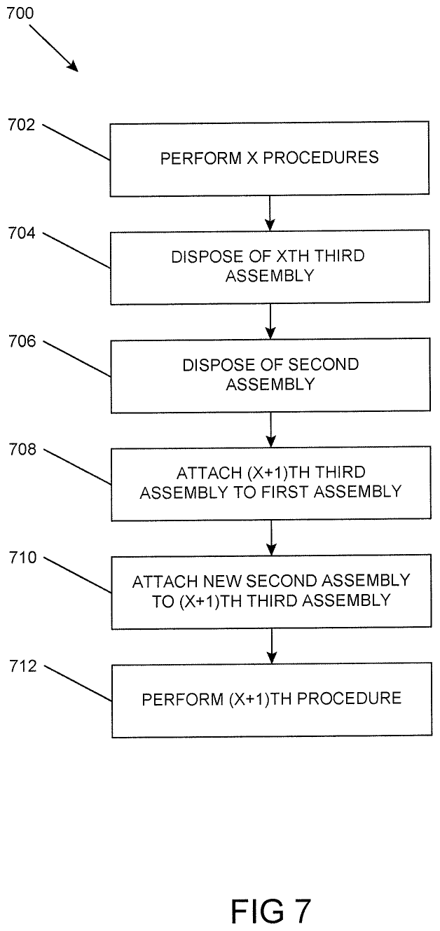

FIG. 7 is a flowchart illustrating a method 700 for assembling a robotic introducer system to perform an operation, in accordance with an embodiment. When describing the method 700, reference is made to FIGS. 1 and 6.

At block 702, X procedures are performed, where X is an integer greater than 0. Each procedure of the X procedures can be performed in accordance with one or more steps of the method 600 described above. Accordingly, each procedure of the X procedures can include replacing a third assembly 16 with a different third assembly 16, e.g., a new third assembly 16. The second assembly 14 is constructed and arranged for reuse after each procedure of the X procedures. After each use, the second assembly 14 is sanitized as described herein.

At block 704, an Xth third assembly is removed from the robotic introducer system 10 and disposed of.

At block 706, the second assembly 14 is disposed of after X procedures are performed.

At block 708, a new third assembly, i.e., an (X+1)th third assembly, is attached to the first assembly 12.

At block 710, a new second assembly is attached to the (X+1)th third assembly. Accordingly, the robotic introducer system 10 is operational.

At block 712, an (X+1) procedure can be performed by the robotic introducer system 10, for example, a medical procedure.

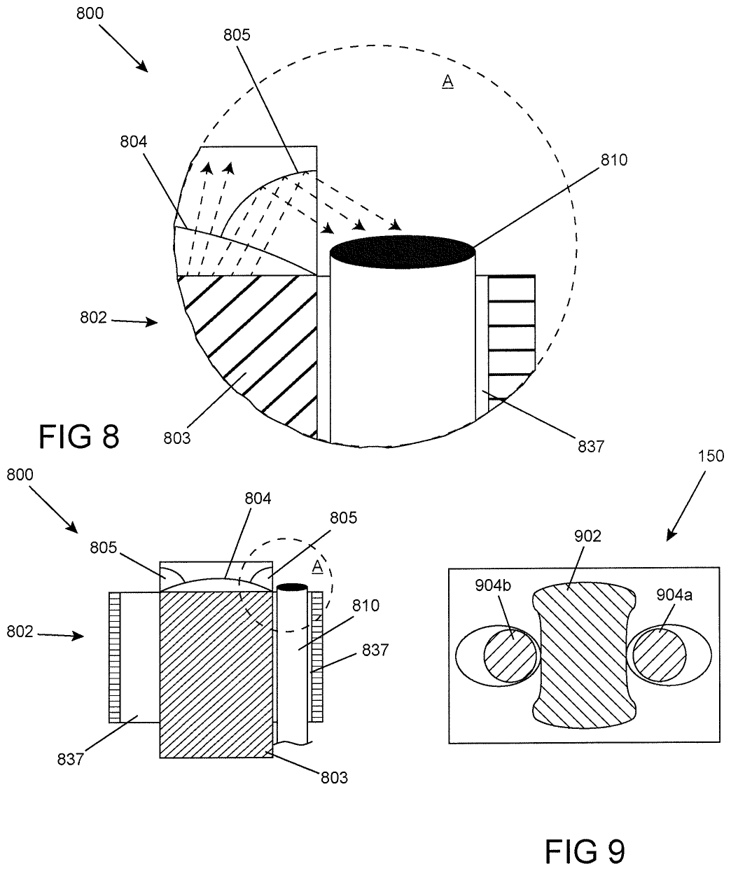

FIG. 8 is a cross-sectional view of an optical assembly 800, in accordance with an embodiment. The optical assembly 800 can be constructed and arranged to be part of a distal link extension assembly 802 coupled to a distal end of a probe assembly, for example, the articulating probe assembly 120 described with reference to FIGS. 1-5. The distal link extension assembly 802 can be similar to the distal link extension assembly 202 described with reference to FIGS. 1-5. Repetitive details of the distal link extension assembly 802 will not be repeated for brevity. The optical assembly 800 can include elements similar to the camera assembly 304 and/or the lighting assembly 306 described with reference to FIGS. 1-5. Accordingly, details will not be repeated for brevity

The distal link extension assembly 802 can include a distal link body 803. At least one side port 837 extends from the distal link body 803. The side port 837 is constructed and arranged to receive a tool 810, for example, a cutter, a grasper, an energy delivery probe, a lighting fiber, etc. The optical assembly 800 can include a lens 804 that provides a first field of view, for example, collects images taken during a procedure. The optical assembly 800 can include an optical redirector 805 such as a mirror or prism that is adjacent the lens 804, and positioned so that an output of the lens 804, for example, optical pathways, are reflected from the optical redirector. For example, some of the optical pathway is directed towards the optical redirector 805, where it is then redirected toward the side port 837.

In this manner, the optical element 805 provides a second field of view that complements the first field of view of the lens 804. The combination of the lens 804 and the optical element 805 can provide a combined field of view that is up to 180°, and in some cases, greater than 180°. This feature permits an operator to view multiple images proximate distal link extension assembly 802. For example, as shown in FIG. 9, console system 150 can produce multiple images 902, 904a and 904b. Image 902 represents an image of a region in front of lens 804. Image 904a and 904b represent images of the tool ports 837 on either side of lens 804. In some embodiments, the optical element 805 is configured to allow viewing of a tool initially exiting a side port 837, where an image can be outside of the first field of view of lens 804, such as tool 810 shown.

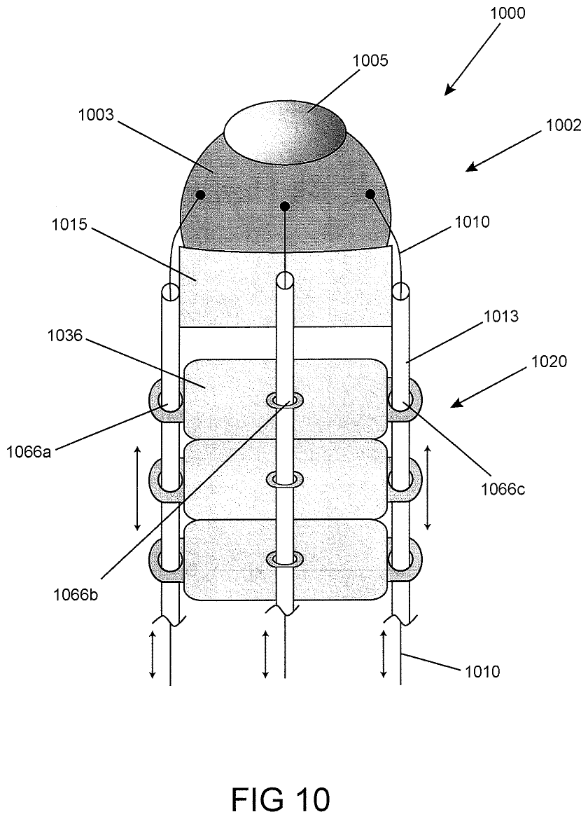

FIG. 10 is a cross-sectional view of a robotic introducer system 1000 comprising a distal link extension assembly 1002, in accordance with an embodiment. The distal link extension assembly 1002 is coupled to a distal end of an articulating probe assembly 1020. The probe assembly 1020 can include elements that are the same as or similar to the articulating probe assembly 120 described herein, and will not be repeated for brevity.

The distal link extension assembly 1002 includes a base 1015, a body 1003 movably positioned in the base 1015, and an optical lens 1005 coupled to the body 1003. A plurality of body articulating cables 1010 extend along the probe assembly 1020 and the base 1015. A distal end of each articulating cable 1010 is attached to the body 1003. The articulating cables 1010 can be advanced or retracted in response to a force applied to the cables 1010 to move the body 1003 for changing a field of view of the lens 1005. The articulating probe assembly 1020 and the body 1003 are independently controllable. For example, the articulating cables 1010 can be advanced and retracted to move the body 1003 relative to an axis along which the robotic introducer system 1000 extends while the articulating probe assembly 1020 remains stationary along the axis.

The articulating probe assembly 1020 includes a plurality of probe links, for example, inner probe links and outer probe links similar to the probe assembly 120 described above and/or described in PCT Application No. PCT/US2012/032279, filed Apr. 5, 2012, the content of which is incorporated herein by reference above. The distal link extension assembly 1002 is adjacent a distal link 1036 of the probe links. The articulating probe assembly 1020 can include at least one steering cable that extends through the links and terminates at the distal link 1036. The steering cable and the body articulating cables 1010 are independently controllable.

The base 1015 can include a concave region, which can mate with a convex lower region of the body 1003. In an embodiment, the body 1003 is ball-shaped and is positioned in a cavity of the base 1015. Alternatively, the base 1015 can include a convex region, which can mate with a concave lower region of the body 1003. The coupling of the base 1015 and the body 1003 in this manner permits a rotation of the body 1003 relative to the base 1015 in response to a force applied to the body articulating cables 1010. The body 1003 and/or the base 1015 can have a cavity or a protruding region having other shapes, for example, semi-spherical, semi-ellipsoidal, or parabolic shape.

A plurality of guide holes 1066 (1066a-c shown) can extend from the probe assembly 1020. The articulating body cables 1010 can extend through the guide holes 1066. In an embodiment, each link 1036 in the articulating probe assembly 1020 has a guide hole 1066a, 1066b, 1066c (generally, 1066). Two or more guide holes 1066, for example, guide holes 1066a, 1066c can be aligned with each other to receive an articulating body cable 1010. A plurality of flexible tubes 1013 can extend through the guide holes 1066 along the probe assembly 1020. The tubes 1013 can be spaced equidistantly with respect to each other about the probe assembly 1020. The tubes 1013 can advance and retract with respect to the probe assembly for articulating the probe assembly 1020. The tubes 1013 can move in concert with, or independently of, a movement of the steering cables (not shown) extending through an interior of the links 1036. The body articulating cables 1010 and the tubes 1013 can operate to pan or tilt, the lens 1005 coupled to the body 1003. Alternatively or additionally, body articulating cables 1010 and the tubes 1013 can operate to zoom lens 1005 (e.g. by advancing the body 1003). A distal end of each of the tubes 1013 is coupled to the base 1015. A body articulating body cable 1010 extends through each tube 1013.

The lens 1005 can be part of a camera assembly, for example, a camera assembly described herein, such as a camera assembly contained in whole or in part in body 1003. Details of the camera assembly are not repeated for brevity. The body 1003 can include a hollow interior or include a cavity in which the camera assembly can be positioned. The lens 1005 is positioned at a top region of the body 1003 for providing a field of view.

While the present inventive concepts have been particularly shown and described above with reference to exemplary embodiments thereof, it will be understood by those of ordinary skill in the art, that various changes in form and detail can be made without departing from the spirit and scope of the present inventive concepts described and defined by the following claims.

What is claimed is:

1. A robotic introducer system, comprising:
a first assembly comprising a cable control assembly, the first assembly constructed and arranged for use in a plurality of medical procedures;
a second assembly comprising a distal link extension assembly, the second assembly constructed and arranged for fewer uses than the first assembly; and
a third assembly coupled between the first and second assemblies, the third assembly comprising an articulating probe assembly to which the distal link extension assembly is removably coupled, and which is controlled by the cable control assembly, the third assembly constructed and arranged for fewer uses than the second assembly.
